(19) United States
(12) Patent Application Publication
(10) Pub. No.: US 2013/0131003 A1
(43) Pub. Date: May 23, 2013

(54) ORALLY DISINTEGRATING TABLET CONTAINING ACARBOSE

(71) Applicants: Axel Schneeweis, Berlin (DE); Tobias Laich, Koln (DE)

(72) Inventors: Axel Schneeweis, Berlin (DE); Tobias Laich, Koln (DE)

(73) Assignee: BAYER INTELLECTUAL PROPERTY GMBH, Monheim (DE)

(21) Appl. No.: 13/643,929

(22) PCT Filed: Apr. 26, 2011
(86) PCT No.: PCT/EP2011/056587

§ 371 Date: Feb. 4, 2013

(30) Foreign Application Priority Data
Apr. 27, 2010 (EP) 10 161 114.3

(51) Int. Cl. A61K 47/00

(52) U.S. Cl. CPC A61K 47/00
USPC 514/42

(57) ABSTRACT

It was an object of the present invention to provide an orally disintegrating tablet (ODT) for the glycosidase inhibitor acarbose. The object is achieved with an orally disintegrating tablet containing 1-30% acarbose and 40-90% water-soluble carrier. In order to obtain the desired properties, the ingredients have to be precompacted and to be premixed with an insoluble carrier.

It was an object of the present invention to provide an orally disintegrating tablet (ODT) for the glycosidase inhibitor acarbose. The object is achieved with an orally disintegrating tablet containing 1-30% acarbose and 40-90% water-soluble carrier. In order to obtain the desired properties, the ingredients have to be precompacted with an insoluble lubricant and to be premixed with a water-insoluble carrier.

Optimal action of glycosidase inhibitors as antidiabetic is aided by as uniform a distribution as possible of the active ingredient in the ingested food. Such a uniform distribution can be achieved with the aid of an orally disintegrating tablet. The tablet and the active ingredient dissolves in the mouth, and the active ingredient is swallowed as a solution and comes, in the stomach, to the ingested food as a solution and can easily distribute therein.

The preparation of orally disintegrating tablets of the active ingredient acarbose is problematic, since the active ingredient results in very hard, slow-dissolving tablets owing to its physicochemical properties. A fast-dissolving orally disintegrating tablet can be obtained when a large portion (<50%) of water-insoluble carriers is introduced into the tablet. The mouthfeel of these tablets is, however, not satisfactory, since the large proportion of insoluble excipients on the tongue is perceived as a rough foreign substance.

The development work for the present invention therefore concentrated on formulations having a low water-insoluble proportion.

By selecting suitable excipients and a suitable process (precompacting of acarbose), formulations were found which both feel pleasant in the mouth and release very rapidly.

The object was achieved by the formulations presented below and the associated process:

The formulation according to the invention is an orally disintegrating tablet containing 1-30% acarbose and 40-90% water-soluble carrier. It has a disintegration time of less than 60 sec, preferably less than 45 sec, more preferably less than 30 sec, even more preferably less than 20 sec The water-soluble carrier is the product Ludiflash®. Ludiflash® is composed of the following: 90% mannitol, 5% crospovidone, and 5% polyvinyl acetate. Likewise, it is possible to use as a water-soluble carrier, optionally in a mixture with binders: mannitol, isomalt, sorbitol, lactose, starch, modified starch, and maltodextrin. For the properties and rapid solubility, it is important that the overall moisture of the orally disintegrating tablet is between 0-8%, preferably between 1-5%. The tablets have an abrasion of below 1% and have a breaking strength which is between 20-50 N, preferably between 25-45 N. Before tableting, the acarbose is brought to an average particle size of 100 to 800 μm, preferably between 100-600 μm.

### EXAMPLES

Formulation 1

| | Amount [mg] |
|---|---|
| Constituents | |
| Acarbose | 50 000 |
| Ludiflash ® | 111 100 |
| Microcrystalline cellulose | 67 650 |
| Crospovidone | 12 500 |
| Citric acid | 2500 |
| Apple aroma | 2500 |
| Green dye | 1250 |
| Magnesium stearate | 2500 |
| Weight | 250 000 |

Formulation 2

| | Amount [mg] |
|---|---|
| Constituents | |
| Acarbose | 100 000 |
| Ludiflash ® | 222 200 |
| Microcrystalline cellulose | 135 300 |
| Crospovidone | 25 000 |
| Citric acid | 5000 |
| Apple aroma | 5000 |
| Green dye | 2500 |
| Magnesium stearate | 5000 |
| Weight | 500 000 |

Formulation 3

| | Amount [mg] |
|---|---|
| Constituents | |
| Acarbose | 50 000 |
| Ludiflash ® | 111 100 |
| Microcrystalline cellulose | 67 650 |
| Crospovidone | 12 500 |
| Citric acid | 2500 |
| Apple aroma | 2500 |
| Green dye | 1250 |
| Sodium stearyl fumarate | 2500 |
| Weight | 250 000 |

Formulation 4

| | Amount [mg] |
|---|---|
| Constituents | |
| Acarbose | 100 000 |
| Ludiflash ® | 222 200 |
| Microcrystalline cellulose | 135 300 |
| Crospovidone | 25 000 |
| Citric acid | 5000 |
| Apple aroma | 5000 |
| Green dye | 2500 |
| Sodium stearyl fumarate | 5000 |
| Weight | 500 000 |

Formulation 5

| | Amount [mg] |
|---|---|
| Constituents | |
| Acarbose | 50 000 |
| Ludiflash ® | 111 100 |
| Microcrystalline cellulose | 67 650 |
| Croscarmellose sodium | 12 500 |
| Citric acid | 2500 |
| Apple aroma | 2500 |
| Green dye | 1250 |
| Magnesium stearate | 2500 |
| Weight | 250 000 |

Formulation 6

| | Amount [mg] |
|---|---|
| Constituents | |
| Acarbose | 100 000 |
| Ludiflash ® | 222 200 |
| Microcrystalline cellulose | 135 300 |
| Croscarmellose sodium | 25 000 |
| Citric acid | 5000 |
| Green dye | 5000 |
| Magnesium stearate | 5000 |
| Weight | 500 000 |

In the first step of the preparation, the acarbose is granulated with a lubricant; then the granulated substance is mixed with microcrystalline cellulose, such as Avicel for example. The granulation is achieved preferably by means of dry granulation. For this purpose, use is made of, for example, roller compactors, in which the powder is metered through a defined, narrow gap between two rotating rollers and is compressed by pressure alone to form flat, elongated strands, known as ribbons. These ribbons have to be reduced in size in a subsequent step so that they can be metered directly into a tablet press. The preferred average particle size of the compact is between 100 and 800 μm, preferably between 100-600 μm. Most preferably, use is made of a compact having a particle size of at least 15%>250 μm.

After admixing further excipients, an orally disintegrating tablet containing 1-30% acarbose and 40-90% water-soluble carrier and 1-50% water-insoluble carrier is then prepared from this compact by means of tableting. By precompacting the acarbose and subsequently admixing the components, the contact area between the acarbose and the excipients required for the disintegration is minimized. Therefore, the tablets prepared in this way have a disintegration time of less than 60 sec, preferably less than 45 sec, more preferably less than 30 sec, even more preferably less than 20 sec. The overall moisture of the orally disintegrating tablets is between 0 and 8%, preferably between 1 and 5%. The invention also relates to a process for preparing orally disintegrating tablets containing acarbose and further excipients, comprising the steps
1.) precompacting acarbose
2.) mixing with water-insoluble carriers, such as microcrystalline cellulose for example
3.) mixing with water-soluble carrier and with subsequent
4.) tableting.
Optionally, point 2 and 3 can be combined.

Use is made of acarbose having a moisture content of between 0 and 5%, preferably between 1 and 4%. The preferred average particle size of the acarbose compact is between 1 and 200 μm. The tablets have an abrasion of below 1% and have a breaking strength which is between 10-50 N, preferably between 15-45 N. Most preferably, use is made of an acarbose compact having a particle size of 15%>250 μm.

It is common to all the formulations that the acarbose is not processed in a pure form with the water-soluble filler. Using a pure form leads to hard tablets. By enveloping with Avicel in an intermediate step, a rapid disintegration of the tablet is also achieved with the addition of a water-soluble filler. An advantage of the water-soluble filler is the better mouthfeel of the formulation, and also the better stability with respect to the disintegration time of the tablet. The tablets are characterized by the stability being at least 2 years, preferably 3 years.

### Example

### Determining the Disintegration Time of Tablets Comprising Pure Acarbose and Precompacted Acarbose

| | | Acarbose, |
|---|---|---|
| | | precompacted |
| Disintegration[s] | Acarbose, pure | particles |
| Start | 13 s | 9 s |
| 6 weeks, 25° | 13 s | 7 s |
| 6 weeks, 40° | 41 s | 12 s |
| 12 weeks, 25° | 17 s | 11 s |
| 12 weeks, 40° | 43 s | 15 s |

1. Orally disintegrating tablet containing 1-30% acarbose, 40-90% water-soluble carrier, and 1-50% water-insoluble carrier.

2. Tablet according to claim 1 having a disintegration time of less than 60 sec.

3. Tablet according to claim 1 having an overall moisture between 0 and 8%.

4. Tablet according to claim 1 having a breaking strength of 10-50 N.

5. Process for preparing orally disintegrating tablets containing acarbose, comprising the steps
a) precompacting acarbose
c) mixing with water-insoluble carriers
c) mixing with water-soluble carrier
d) tableting, characterized in that acarbose having a moisture content of between 0 and 5% is used.

6. Process according to claim 5, characterized in that acarbose having a mean particle size of 100 to 600 μm is used.

7. Orally disintegrating tablet according to claim 1 for the treatment of diabetes mellitus.
